(19) United States
(12) Patent Application Publication
(10) Pub. No.: US 2024/0415375 A1
(43) Pub. Date: Dec. 19, 2024

(54) ENDOSCOPE WITH INTEGRATED STABILIZER AND CANNULATION ELEMENTS

(71) Applicant: GYRUS ACMI, INC. D/B/A OLYMPUS SURGICAL TECHNOLOGIES AMERICA, Westborough, MA (US)

(72) Inventor: Nikhil M. Murdeshwar, Maple Grove, MN (US)

(21) Appl. No.: 18/700,815

(22) PCT Filed: Oct. 4, 2022
(86) PCT No.: PCT/US2022/077539

§ 371 Date: Apr. 12, 2024

Related U.S. Application Data
(60) Provisional application No. 63/270,696, filed on Oct. 22, 2021.
(60) Provisional application No. 63/267,657, filed on Feb. 7, 2022.

(51) Int. Cl. A61B 1/012; A61B 1/00; A61B 1/005; A61B 1/01; A61B 1/04; A61B 1/273

(52) U.S. Cl. CPC A61B 1/0125; A61B 1/00148; A61B 1/0055; A61B 1/01; A61B 1/04; A61B 1/273

(57) ABSTRACT

An endoscope system can include a control handle, a primary scope, an auxiliary scope, and a guide. The primary scope can extend from the control handle to a distal portion and can be configured for insertion into a passage of a patient, the primary scope can define a working channel extending therethrough. The auxiliary scope can be located at least partially within the working channel and can be movable to extend from the primary scope. The guide can be extendable from the distal portion of the primary scope to guide extension of the auxiliary scope from the primary scope.

### PRIORITY CLAIM

This application claims the benefit of priority to U.S. Provisional Patent Application Ser. No. 63/270,696, filed Oct. 22, 2021, and U.S. Provisional Patent Application Ser. No. 63/267,657, filed Feb. 7, 2022; the contents of which are hereby incorporated by reference in their entireties.

### TECHNICAL FIELD

The present disclosure relates generally to medical devices and instruments configured to provide diagnostic and treatment operations. More specifically, the present disclosure relates to medical device systems comprising elongate bodies, such as endoscopes, that can be inserted into incisions or openings in anatomy of a patient and then advanced to reach locations deep within anatomic passageways of the patient where the diagnostic and treatment operations can be performed.

### BACKGROUND

Endoscopes can be used to provide passage of other devices, e.g., therapeutic devices or tissue collection devices, toward various anatomical portions, or for imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Endoscopic retrograde cholangiopancreatography (ERCP) and cholangioscopy procedures can be performed to diagnose or treat the common bile duct. These processes often use two scopes. One scope (e.g., duodenoscope) to first navigate upper gastrointestinal anatomy to the ampulla of Vater. Then, a second scope (e.g., a cholangioscope) can be advanced through the first scope to cannulate the common bile duct.

### SUMMARY

In an endoscopy, a distal portion of the endoscope can be configured for supporting and orienting a therapeutic device, such as with the use of an elevator. In some systems, two endoscopes can be configured to work together with a first (or primary) scope guiding a second (or auxiliary) scope inserted therein with the aid of the elevator for positioning of the second scope. Such systems can be helpful in guiding endoscopes to anatomic locations within the body that are difficult to reach. For example, some anatomic locations can only be accessed with an endoscope after insertion through a circuitous path. However, such systems can be relatively complex and can be difficult to use or can require significant training to become proficient.

The present disclosure helps to address these issues by providing a scope system that can include a guide for guiding extension of a second scope from a primary scope. The guide can be supported by an arm to help ensure that the guide extends properly from the primary scope. The guide can also be used for cannulation, helping to reduce a number of components required. Once extended, the guide can be used to engage the auxiliary scope as the auxiliary scope extends from a working channel of the primary scope, which can help guide the auxiliary scope in a proper direction and ultimately to its desired location, such as a sphincter of Oddi. Further, an imaging device can be located on an outer surface of the primary scope for improved visibility and reduced interference with the guide and the second (auxiliary scope). Moreover, projections can be extendable from the primary scope to engage the passage (e.g., duodenum) to help stabilize the distal end of the scope system during the procedure, further helping to increase accuracy, precision, and ease of use of the scope system.

The above discussion is intended to provide an overview of subject matter of the present patent application. It is not intended to provide an exclusive or exhaustive explanation of the invention. The description below is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of an endoscopy system.

FIG. 2 illustrates a schematic diagram of the imaging and control system of FIG. 1 showing the imaging and control system connected to the endoscope.

FIG. 3 illustrates a schematic view of a distal portion of an endoscope in use.

FIG. 4 illustrates a perspective view of a portion of an endoscope in a first condition.

FIG. 5 illustrates a perspective view of a portion of an endoscope in a second condition.

FIG. 6 illustrates a perspective view of a portion of an endoscope in a third condition.

FIG. 7 illustrates a perspective view of a portion of an endoscope in a fourth condition.

FIG. 8 illustrates an end view of a portion of an endoscope system.

FIG. 9 illustrates an end view of a portion of an endoscope system.

FIG. 10 illustrates an side view of a portion of an endoscope system.

FIG. 11 illustrates a schematic view of a method of using a endoscope system.

FIG. 12 illustrates a block diagram illustrating an example of a machine upon which one or more embodiments may be implemented.

### DETAILED DESCRIPTION

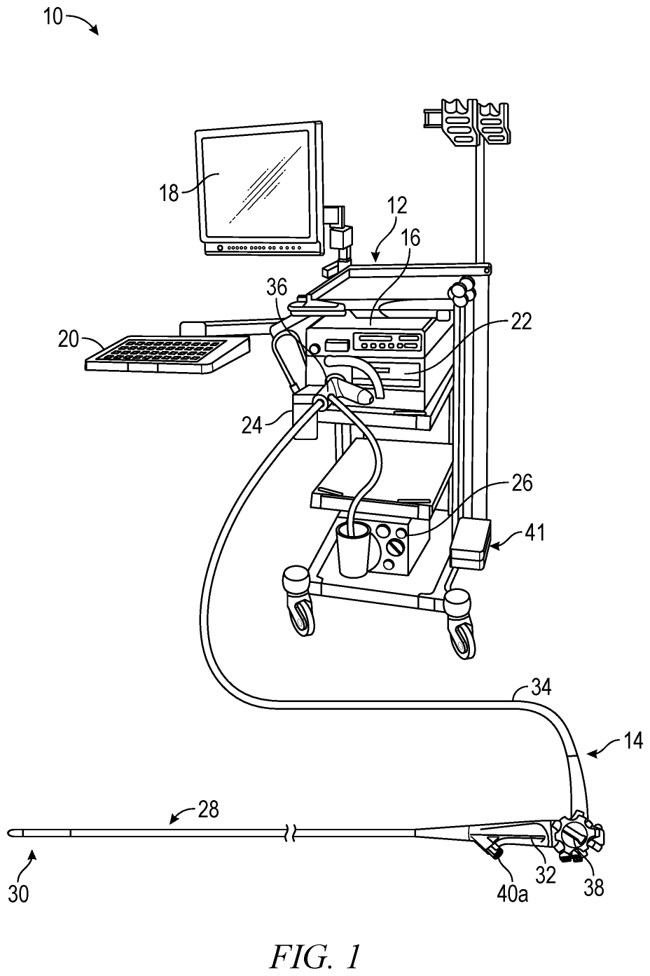

FIG. 1 is a schematic diagram of an endoscopy system 10 that can include an imaging and control system 12 and an endoscope 14. The system of FIG. 1 is an illustrative example of an endoscopy system suitable for use with the systems, devices and methods described herein, such as an endoscope with an integrated guide and arm for guiding extension of an auxiliary scope.

The endoscope 14 can be insertable into an anatomical region for imaging or to provide passage of or attachment to (e.g., via tethering) one or more sampling devices for biopsies, or one or more therapeutic devices for treatment of a disease state associated with the anatomical region. The endoscope 14 can interface with and connect to an imaging and control system 12. The endoscope 14 can also include a duodenoscope, though other types of endoscopes can be used with the features and teachings of the present disclosure. The imaging and control system 12 can include a control unit 16, an output unit 18, an input unit 20, a light source 22, a fluid source 24, and a suction pump 26.

The imaging and control system 12 can include various ports for coupling with the endoscopy system 10. For example, the control unit 16 can include a data input/output port for receiving data from and communicating data to the endoscope 14. The light source 22 can include an output port for transmitting light to the endoscope 14, such as via a fiber optic link. The fluid source 24 can include a port for transmitting fluid to the endoscope 14. The fluid source 24 can include, for example, a pump and a tank of fluid or can be connected to an external tank, vessel or storage unit. The suction pump 26 can include a port used to draw a vacuum from the endoscope 14 to generate suction, such as for withdrawing fluid from the anatomical region into which the endoscope 14 is inserted. The output unit 18 and the input unit 20 can be used by an operator of the endoscopy system 10 to control functions of the endoscopy system 10 and view output of the endoscope 14. The control unit 16 can additionally be used to generate signals or other outputs from treating the anatomical region into which the endoscope 14 is inserted. In some examples, the control unit 16 can generate electrical output, acoustic output, a fluid output and the like for treating the anatomical region with, for example, cauterizing, cutting, freezing and the like.

The endoscope 14 can include an insertion section 28, a functional section 30 and a handle section 32, which can be coupled to a cable section 34 and a coupler section 36. The coupler section 36 can be connected to the control unit 16 to connect the endoscope 14 to multiple features of the control unit 16, such as the input unit 20, the light source unit 22, the fluid source 24, and the suction pump 26.

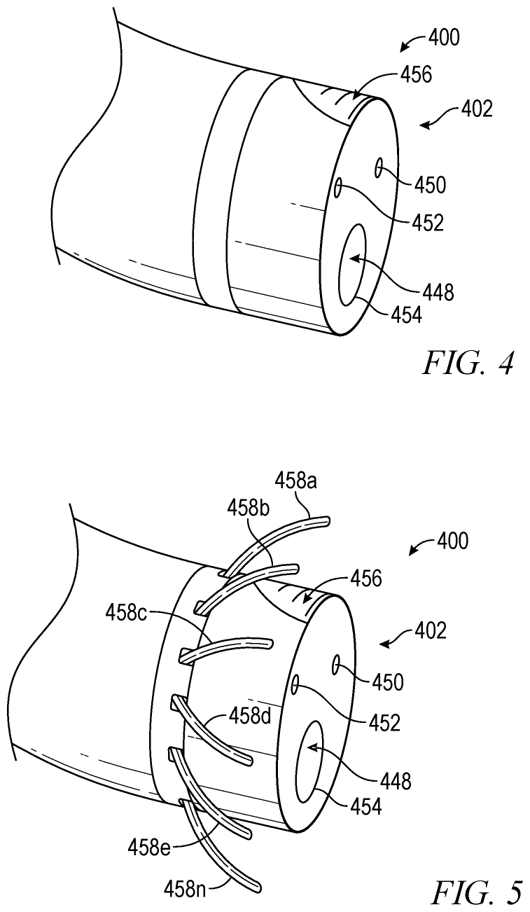

The insertion section 28 can extend distally from the handle section 32 and the cable section 34 can extend proximally from the handle section 32. The insertion section 28 can be elongate and include a bending section, and a distal end to which the functional section 30 can be attached. The bending section can be controllable (e.g., by a control knob 38 on the handle section 32) to maneuver the distal end through tortuous anatomical passageways (e.g., stomach, duodenum, kidney, ureter, etc.). The insertion section 28 can also include one or more working channels (e.g., an internal lumen) that can be elongate and can support insertion of one or more therapeutic tools of the functional section 30, such as an auxiliary scope 134 (of FIG. 4). The working channel can extend between the handle section 32 and the functional section 30. Additional functionalities, such as fluid passages, guide wires, and pull wires can also be provided by the insertion section 28 (e.g., via suction or irrigation passageways, or the like).

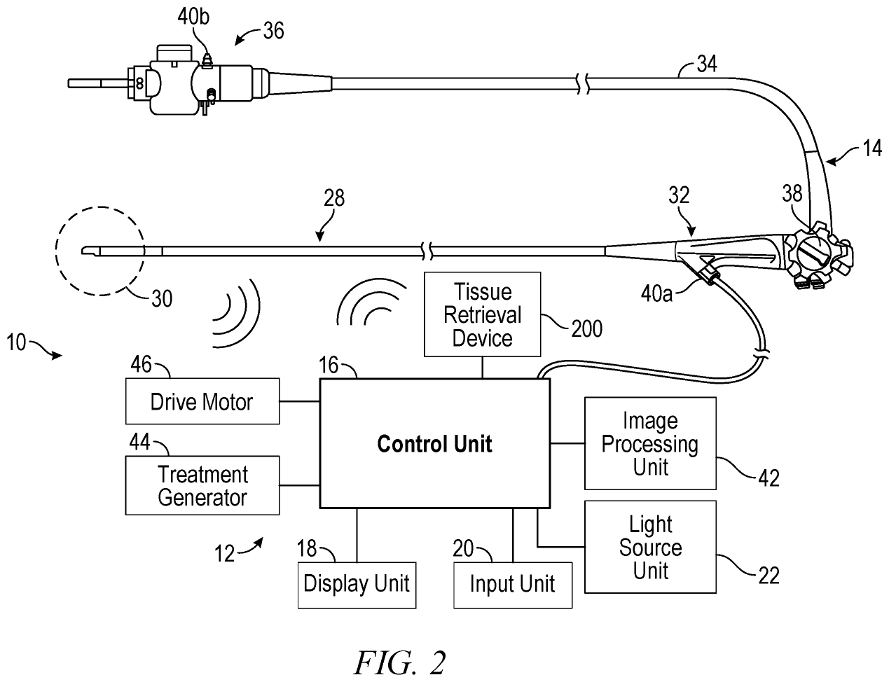

The handle section 32 can include the knob 38 as well as a port 40a. The knob 38 can be connected to a pull wire, or other actuation mechanisms, extending through insertion the section 28. Port 40a, as well as other ports, such as the port 40B (of FIG. 2) can be configured to couple various electrical cables, guide wires, auxiliary scopes, tissue collection devices of the present disclosure, fluid tubes and the like to the handle section 32, such as for coupling with the insertion section 28.

The imaging and control system 12 can be provided on a mobile platform (e.g., a cart 41) with shelves for housing the light source 22, the suction pump 26, an image processing unit 42 (FIG. 2), etc. Alternatively, several components of imaging and the control system 12 shown in FIGS. 1 and 2 can be provided directly on the endoscope 14 so as to make the endoscope self-contained.

The functional section 30 can include components for treating and diagnosing anatomy of a patient. The functional section 30 can include an imaging device, an illumination device and a guide, as discussed in further detail below. The functional section 30 can further include optically enhanced biological matter and tissue collection and retrieval devices as are described herein. For example, the functional section 30 can include one or more electrodes conductively connected to the handle section 32 and functionally connected to the imaging and control system 12 to analyze biological matter in contact with the electrodes based on comparative biological data stored in the imaging and control system 12.

FIG. 2 is a schematic diagram of the endoscopy system 10 of FIG. 1 including the imaging and control system 12 and the endoscope 14. FIG. 2 schematically illustrates components of the imaging and the control system 12 coupled to the endoscope 14, which in the illustrated example includes a duodenoscope. The imaging and control system 12 can include the control unit 16, which can include or be coupled to an image processing unit 42, a treatment generator 44 and a drive unit 46, as well as the light source 22, the input unit 20, and the output unit 18. The control unit 16 can include, or can be in communication with, an endoscope 100, a surgical instrument and an endoscopy system, which can include a device configured to engage tissue and collect and store a portion of that tissue and through which imaging equipment (e.g., a camera) can view target tissue via inclusion of optically enhanced materials and components. The control unit 16 can be configured to activate a camera to view target tissue distal of a surgical instrument and the endoscopy system. Likewise, the control unit 16 can be configured to activate the light source unit 22 to shine light on the surgical instrument, which can include select components that are configured to reflect light in a particular manner, such as tissue cutters being enhanced with reflective particles.

The image processing unit 42 and light source 22 can each interface with the endoscope 14 (e.g., at the functional unit 30) by wired or wireless electrical connections. The imaging and control system 12 can accordingly illuminate an anatomical region, collect signals representing the anatomical region, process signals representing the anatomical region, and display images representing the anatomical region on the display unit 18. The imaging and control system 12 can include light the source 22 to illuminate the anatomical region using light of desired spectrum (e.g., broadband white light, narrow-band imaging using preferred electromagnetic wavelengths, and the like). The imaging and control system 12 can connect (e.g., via an endoscope connector) to the endoscope 14 for signal transmission (e.g., light output from light source, video signals from imaging system in the distal end, diagnostic and sensor signals from a diagnostic device, and the like).

The fluid source 24 (shown in FIG. 1) can be in communication with control unit 16 and can include one or more sources of air, saline or other fluids, as well as associated fluid pathways (e.g., air channels, irrigation channels, suction channels) and connectors (barb fittings, fluid seals, valves and the like). The fluid source 24 can be utilized as an activation energy for a biasing device or a pressure-applying device of the present disclosure. The imaging and control system 12 can also include the drive unit 46, which can include a motorized drive for advancing a distal section of endoscope 14.

The coupler section 36 can be connected to the control unit 16 to connect to the endoscope 14 to multiple features of the control unit 16, such as the image processing unit 42 and the treatment generator 44. In examples, the port 40a can be used to insert another instrument or device, such as a daughter scope or auxiliary scope, into the endoscope 14. Such instruments and devices can be independently connected to the control unit 16 via the cable 47. In some examples, the port 40B can be used to connect coupler section 26 to various inputs and outputs, such as video, air, light and electric.

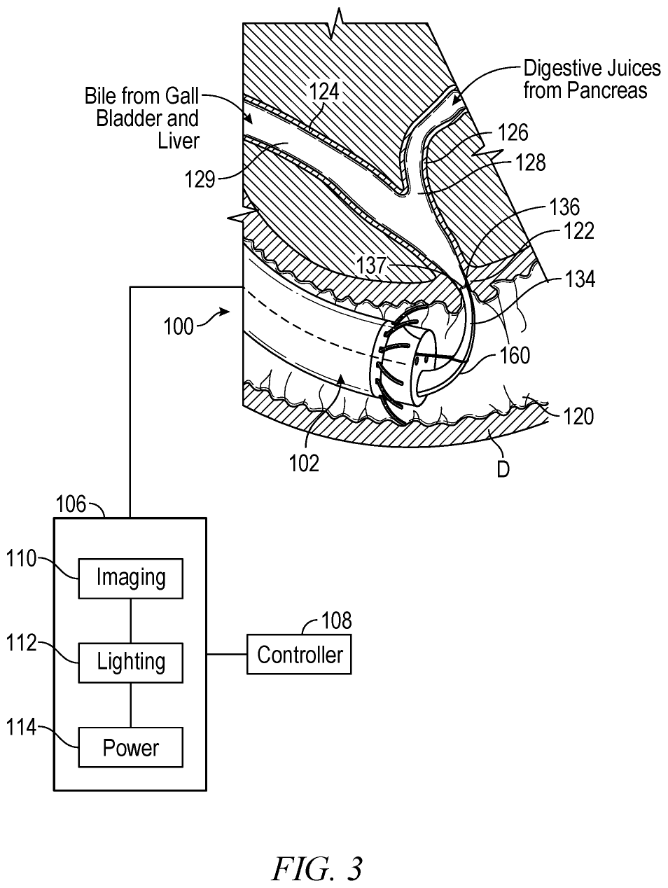

FIG. 3 is a schematic illustration of distal portion of the endoscope 100 according to the present disclosure positioned in duodenum D. The endoscope 100 can include a functional module 102, an insertion section module 104, and a control module 106. The control module 106 can include a controller 108. The control module 106 can include other components, such as those described with reference to the endoscopy system 10 of FIG. 1 and the control unit 16 of FIG. 2. Additionally, the control module 106 can include components for controlling a camera and a light source connected to the auxiliary scope (or daughter scope) 134, such as an imaging unit 110, lighting 112 and a power unit 114. The endoscope 100 can be configured similarly as the endoscope 14 of FIGS. 1 and 2.

Duodenum D can include a duct wall 120, a sphincter of Oddi 122, a common bile duct 124 and a main pancreatic duct 126. The duodenum D includes an upper part of the small intestine. The common bile duct 124 can carry bile from the gallbladder and liver (not illustrated) and empties the bile into the duodenum D through the sphincter of Oddi 122. A main pancreatic duct 126 carries pancreatic juice from the exocrine pancreas to common the bile duct 124. Sometimes it can be desirable to remove biological matter, e.g., tissue, from the bile duct 124 or the pancreatic duct 126 to analyze the tissue to, for example, diagnose diseases or maladies of the patient such as cancer.

The endoscope 100 can further include a lumen 132 and the auxiliary scope 134. The auxiliary scope 134 can include a lumen 136. The auxiliary scope 134 can itself include functional components, such as a camera lens 137 and a light lens coupled to the control module 106, to facilitate navigation of the auxiliary scope 134 from the endoscope 100 through the anatomy and to facilitate viewing of components extending from the lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as the auxiliary scope 134, can be attached and advanced through the lumen 132 (or the central lumen 62 of the insertion section 28 of the endoscope 14 in FIG. 3B) of the “main scope” (also referred to as mother scope, or duodenoscope), such as the endoscope 100. As discussed in greater detail below, the auxiliary scope 134 can be guided into sphincter of the Oddi 122. Therefrom, a surgeon operating the auxiliary scope 134 can navigate the auxiliary scope 134 through the lumen 132 toward the gall bladder, liver, or other locations in the gastrointestinal system to perform various procedures. The surgeon can navigate the auxiliary scope 134 past entry 128 of main the pancreatic duct 126 and into a passage 129 of the common bile duct 124, or into the entry 128. The auxiliary scope 134 can be used to guide an additional device to the anatomy to obtain biological matter, such as by passage through or attachment to the lumen 136. The additional device can have its own functional devices, such as a light source, camera, tissue separators, accessories, and biopsy channel, for therapeutic procedures. Additional devices can include various features, such as forceps or an auger, for gathering biological matter, such as tissue. One or more additional devices can include a biopsy device tethered to the endoscope and that has tissue collection capacity enhancement features. The biological matter can then be removed from the patient, typically by removal of the additional device from the auxiliary device, so that the removed biological matter can be analyzed to diagnose one or more conditions of the patient. According to several examples, the endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like. Further details of the functional module 102 of the endoscope 100 (or devices similar thereto) are discussed below.

FIG. 4 illustrates a perspective view of a functional module 402 of an endoscope 401 of an endoscope system 400 in a first, insertion configuration. FIG. 5 illustrates a perspective view of a functional module 402 of the endoscope 401 in a second, stabilizing configuration. FIGS. 4 and 5 are discussed together below. The functional module 402 of the endoscope 401 can be similar to the functional module 102 of the endoscope 100 discussed above. The functional module 402 can include a working channel 448, a lumen 450, and a light 452.

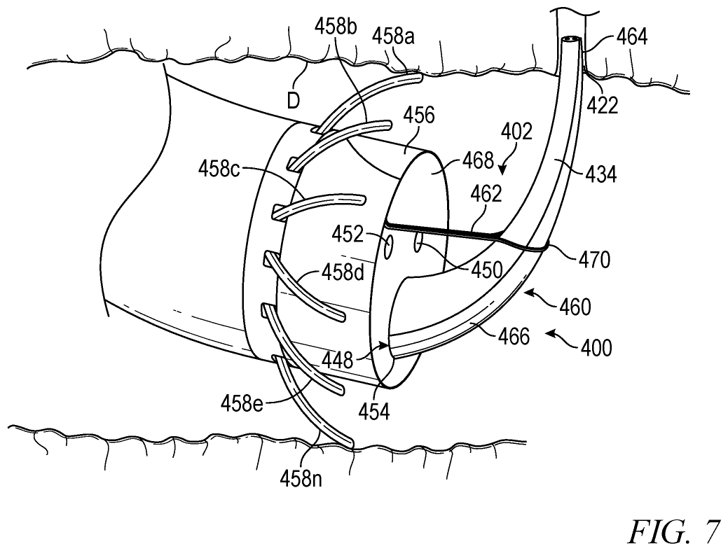

The working channel 448 can be a bore, passage, channel, lumen, or the like, extending through the endoscope 401, such as through the insertion section 28. The working channel 448 can extend through a distal end of the functional module 402 to define an opening 454 via which one or more components can extend. The working channel 448 can be configured to support an instrument therein in addition to an auxiliary or daughter scope, as discussed with respect to FIG. 7.

The lumen 450 can be an additional passage through the endoscope 401, such as through the insertion section 28. The lumen can be or can include a drainage passage or can be configured to receive one or more instruments therein or therethrough. Though one lumen is shown, the functional module 402 can include one or more lumens.

The light 452 can be a light, such as a fiber optic light or light emitting diode (LED), configured to receive power or light from the light source 22 for providing light in the duodenum D ahead of the functional module 402. Though one light is shown, the functional module 402 can include one or more light.

The functional module 402 can also include a camera module 456. The camera module 456 can include a lens, an image capture sensor, and a chip or processor. Optionally, one or more of these components can be located in the image processing unit 42 or can be connected to the image processing unit 42. The camera module 456 can be connected to an outer portion of the functional module 402 of the endoscope 401. Optionally, the camera module 456 can be angled with respect to the outer surface of the functional module 402 or angled with respect to a longitudinal axis of the endoscope 401. By locating the camera module 456 on an outer surface of the functional module 402, the camera module 456 can be clear of the opening 454 and can be in a location to provide an improved viewing angle of anatomy, such as the sphincter of Oddi 122.

FIG. 5 also shows stabilizing features 458a-458n (referred to hereinafter as stabilizers 458) in a deployed position (while FIG. 4 shows the stabilizers 458 in a retracted position). The stabilizers 458 can be pliable or flexible extensions made from one or more of metals, plastics, foams, elastomers, ceramics, composites, or the like. The stabilizers 458 can be movable between the deployed or extended position of FIG. 5 and the retracted position of FIG. 4.

In the retracted position, the stabilizers 458 can be located under a sheath or covering of the endoscope 401. Optionally, the stabilizers 458 can be manipulated to lay flat against the outer surface of the endoscope 401 in the retracted position. Optionally, the stabilizers 458 can remain in an extended position at all times, but can be sufficiently flexible to provide stability for the endoscope 401 without significantly impairing insert ability or navigation of the endoscope 401.

In the extended or deployed position of FIG. 5, the stabilizers 458 can be configured to engage the duodenum D (or any other internal passage) to help limit movement of the endoscope 401 and functional module 402, especially once the functional module 402 reaches a desired location, such as near the sphincter of Oddi 122. The stabilizers 458 can optionally be biased proximally or distally to help limit axial movement of the functional module 402 of the endoscope 401.

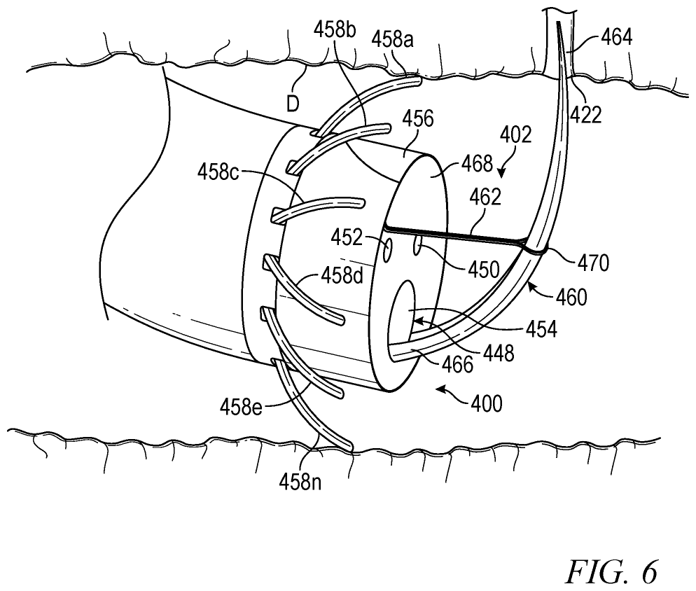

FIG. 6 illustrates a perspective view of a portion of the endoscope 401 with the stabilizers 458 extended and a guide extended. More specifically, FIG. 6 shows that the endoscope 401 can include a guide 460 and an arm 462 to support the guide 460. Further details are discussed below.

The guide 460 can be a pliable or flexible extensions made from one or more of metals, plastics, foams, elastomers, ceramics, composites, or the like. In some examples, the guide 460 can be a thin polymer sheath. The guide 460 can have a relatively small thickness such that the guide 460 can conform to the shape of the objects supporting the guide 460. For example, the curved opening 454 can cause the guide 460 to curve to confirm to the working channel 448.

The guide 460 can include a distal tip 464 and a base portion 466. The guide 460 can be tapered or can decrease in width as it extends distally from a proximal portion to the distal tip 464 such that the distal tip 464 is significantly smaller in width than the base portion 466. The distal tip 464 can be used (e.g., by a user or physician) to cannulate a sphincter of Oddi 422 of the duodenum D. Optionally the distal tip 464 can have an enlarged thickness to help provide some rigidity, such as for improved cannulation performance of the distal tip 464.

The arm 462 can be an arm, lever, holder, or the like movably connected to the functional module 402. For example, the arm 462 can be connected to a distal end 468 of the functional module 402. Optionally, the arm 462 can be connected to the distal end 468 via a hinge such that the arm 462 can move between a stored position and a support position (shown in FIGS. 6 and 7) where the arm 462 is distally extended from the distal end 468.

The 462 can be relatively small such as to limit interference with the lumen 450, light 452, and working channel 448, but can be strong enough to support the guide 460. The arm 462 can include a ring 470 that can be located at a distal end or portion of the arm 462. The ring 470 can be configured to receive the guide 460 therein and can be configured to guide extension of the guide 460 from the working channel 448 of the functional module 402. Optionally, the ring 470 can be configured to stretch to accommodate the guide 460 as it extends through the ring 470. Though the ring 470 is discussed as being a ring, it can have a variety of shapes including open and closed shapes, such as a C-shape, U-shape, D-shape, O-shape, or the like. Further operation of the arm 462 and the guide 460 are discussed below with respect to FIG. 7.

FIG. 7 illustrates a perspective view of the endoscope 401 with the stabilizers 458 extended, with the guide 460 extended, and with an auxiliary scope 434 extended. More specifically, the endoscope system 400 can include the auxiliary scope 434, which can be located at least partially within the working channel 448. The auxiliary scope 434 can be operated, such as from a control handle, to extend from the working channel 448, such as to extend into the sphincter of Oddi 422 for one or more procedures, such as an ERCP procedure.

In operation of the endoscope system 400 of some examples, following stabilization using the stabilizers 458, as described above, the guide 460 can be operated to extend from the opening 454 of the working channel 448 when the arm 462 is in a position where the ring 470 is located near the opening 454. When the distal tip 464 of the guide 460 extends from the opening 454 it can be inserted into and through the ring 470, which can cause the arm 462 to move to the extended position, as shown in FIGS. 6 and 7. The arm 462 can remain in the extended position as the guide 460 continues to extend through the ring 470. Once through the ring 470, the distal tip 464 can be guided to the sphincter of Oddi 422, such as to cannulate the sphincter of Oddi 422. In some examples, the distal tip 464 can be sharp or can deliver energy to ablate tissue to further open or open the sphincter of Oddi 422, such as to perform a Sphincterectomy thereof.

Once the guide 460 is positioned as desired, such as in the sphincter of Oddi 422, the auxiliary scope 434 can be extended from the opening 454 of the working channel 448 to engage the guide 460. The guide 460 can, through such engagement, guide extension of the auxiliary scope 434 from the opening 454 to turn in a direction toward the sphincter of Oddi 422. The auxiliary scope 434 can be guided by the guide 460 to extend into and through the ring 470 of the arm 462. The arm 462 can further guide and support extension of the auxiliary scope 434 from the opening 454 and into the cannulated sphincter of Oddi 422 such as to allow a procedure using the auxiliary scope 434 to be performed.

By using the distal tip 464 to stabilize the functional module 402 and the auxiliary scope 434 with respect to the sphincter of Oddi 422, pancreatitis caused by movement of components can be reduced. The stabilizers 458 can further help to reduce movement to further help to reduce pancreatitis.

Optionally, the auxiliary scope 434 can be integrated into the endoscope 401 to form a single-shaft scope. For example, the auxiliary scope 434 can telescopically extend from the endoscope 401, between 10 and 20 centimeters, such as to perform an ERCP operation or procedure. Following a procedure, the auxiliary scope 434 can retract into the endoscope 401. Optionally, the extension and the retraction of the auxiliary scope 434 can be from the working channel 448. Such a device can help to limit a length of the auxiliary scope 434, which can help to reduce cost and can help save time spent navigating the auxiliary scope 434 through the endoscope 401.

Optionally, the guide 460 can be used for fluoroscopy. For example, the distal tip 464 can include a port, lumen, channel, or the like extending therethrough that can store or deliver a dye into the sphincter of Oddi 422 or any passage or duct.

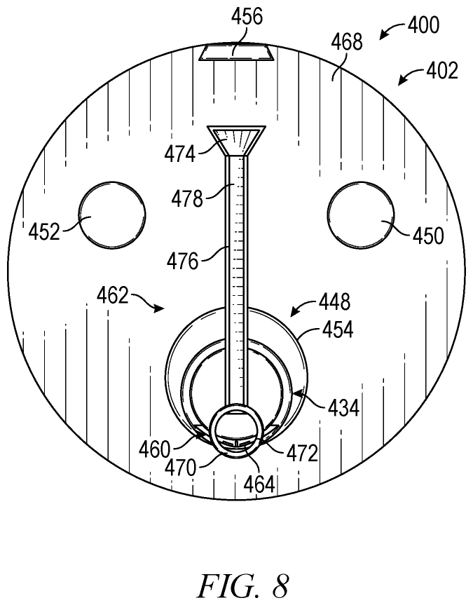

FIG. 8 illustrates an end view of a portion of the endoscope system 400. The endoscope system 400 can be consistent with the endoscope system of FIGS. 4-7 discussed above; FIG. 8 shows additional details of the endoscope system 400.

For example, FIG. 8 shows how an opening 472 of the ring 470 can be aligned with the opening 454 of the working channel 448 when the arm 462 is in a retracted position. In such a position, the distal tip 464 of the guide 460 can be aligned with the ring 470. This alignment can help to ensure that the distal tip 464 extends into and through the opening 472 of the ring 470 as the guide 460 extends from the opening 454 to help ensure that the arm 462 moves to the extended position to properly guide extension or direct advancement of the guide 460 from the working channel 448.

FIG. 8 also shows that the arm 462 can include a base portion 474, a body 476, and a hinge 478. The base portion 474 can be connected to the distal end 468 of the functional module 402 to secure the arm 462 to the functional module 402. A hinge 478 can be connected to the base portion 474 and the body 476 and can be various hinge types, such as a flexible member, living hinge, barrel hinge, flag hinge, pivot hinge, or the like. The hinge 478 can allow the body 476 and the ring 470 to move with respect to the base portion 474 and the distal end 468.

FIG. 8 also shows that the camera module 456 can be located at a top portion of the functional module 402, or a portion opposite the working channel 448, which can help to provide visibility of the guide 460 and the 434 during cannulation or positioning.

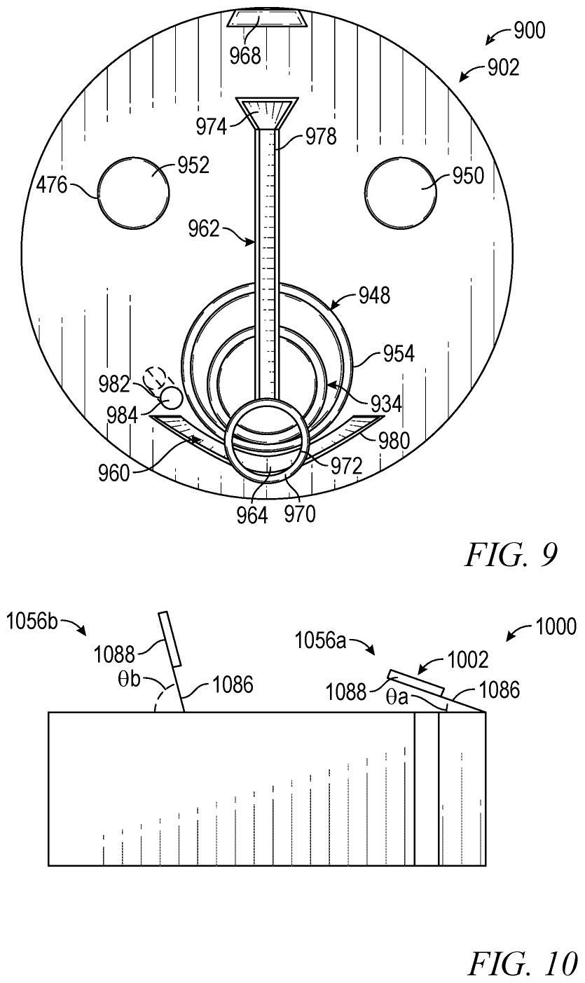

FIG. 9 illustrates an end view of a portion of an endoscope system 900. The endoscope system 900 can be similar to the endoscope system 400 of FIGS. 4-8 discussed above; accordingly, like numerals can represent like components. The endoscope system 900 can differ in that the endoscope system 900 can include a guide wire and the endoscope system 900 can include a guide slot. Any of the systems discussed above or below can be modified to include these features.

More specifically, the endoscope system 900 can include a channel 980 (or passage or slot) located near a working channel 948. The channel 980 can be configured to retain and support a guide 960 in both a retracted state and an extended state. A ring 970 can include an opening 972 configured to align with a distal tip 964 of the guide 960 when the guide 960 is in a retracted state within the channel 980. Including the channel 980 can allow for the guide 960 to be shaped to guide and support extension of an auxiliary scope 934 from the working channel 948. For example, the channel 980 can allow the guide 960 to have a width greater than a diameter of the working channel 948.

FIG. 9 also shows that the endoscope 901 can include a guide wire lumen 982 and a guide wire 984 supported therein or thereby. The guide wire 984 can be movable between a retracted state and an extended state or condition. By using the channel 980 with a relatively wide width, the guide 960 can be used to guide extension of the guide wire 984 from the guide wire channel 982. The ring 970 of an arm 962 can also engage the guide wire 984 to help guide extension of the guide wire 984 from the guide wire channel 982 of the endoscope system 900.

FIG. 10 illustrates a side view of a portion of an endoscope system 1000. The endoscope system 1000 can be similar to the endoscope systems of FIGS. 1-9 discussed above; accordingly, like numerals can represent like components. The endoscope system 1000 can differ in that the endoscope system 1000 can include a camera module at various positions or multiple cameral modules. Any of the systems discussed above or below can be modified to include these features.

FIG. 10 shows that the endoscope system 1000 can include a functional module 1002 including a first camera module 1056a and a second camera module 1056b. The endoscope system 1000 can include both the first camera module 1056a and the second camera module 1056b or can include either of the first camera module 1056a or the second camera module 1056b.

More specifically, FIG. 10 shows that the first camera module 1056a and the second camera module 1056b can each include an arm 1086 and a chip 1088. The chip 1088 can include a lens, image sensor, or the like. Each of the chips 1088 can be connected to the imaging and control system 12.

FIG. 10 further shows how the arms 1086 can be angled. For example, the arm 1086 of the first camera module 1056a can form an angle Θa between the arm 1086 and the functional module 1002. Similarly, the arm 1086 of the second camera module 1056b can form an angle Θb between the arm 1086 and the functional module 1002. The angles Θa and Θb can be different angles or the same. For example, the angle Θa can be relatively small (e.g., 15 degrees) and the angle Θb can be relatively large (e.g., 75 degrees), which can help to provide various viewing angles. Optionally, when the endoscope system 1000 includes multiple modules, the modules can be radially spaced around the functional module 1002 to help limit visual obstruction. Optionally, the modules can be movable between various angles, such as by articulating the arm 1086 of either module.

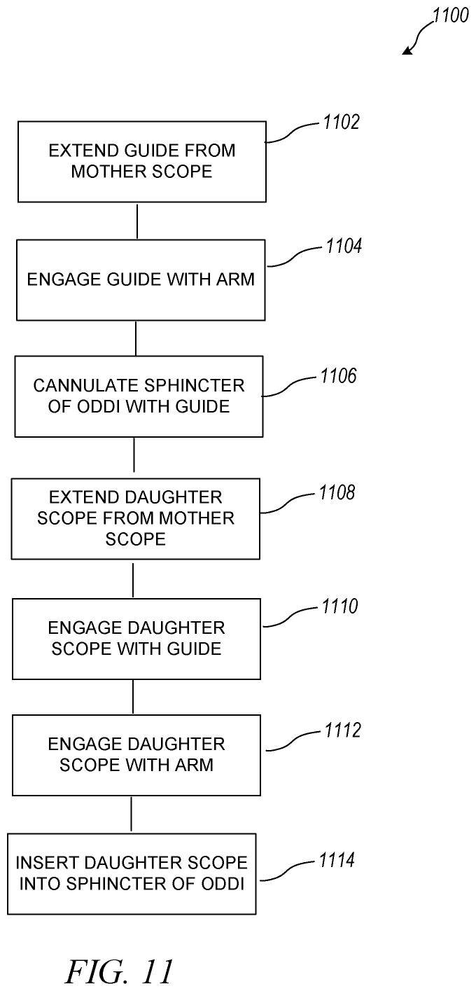

FIG. 11 illustrates a schematic view of the method 1100, in accordance with at least one example of this disclosure. The method 1100 can be a method of operating a scope system. More specific examples of the method 1100 are discussed below. The steps or operations of the method 1100 are illustrated in a particular order for convenience and clarity; many of the discussed operations can be performed in a different sequence or in parallel without materially impacting other operations. The method 1100 as discussed includes operations performed by multiple different actors, devices, and/or systems. It is understood that subsets of the operations discussed in the method 1100 can be attributable to a single actor, device, or system could be considered a separate standalone process or method.

The method 1100 can begin at step 1102, which can be extending a guide from a mother scope. For example, the guide 460 can be extended from the functional module 402 of the endoscope 401. At step 1104, the guide can be engaged with an arm. For example, as the guide 460 extends from the working channel 448 of the functional module 402, the guide 460 can engage the ring 470 of the arm 462 to control or guide extension of the guide 460 from the functional module 402.

At step 1106, the distal tip 464 of the guide 460 can be used to cannulate an opening, such as the sphincter of Oddi 422. Following cannulation, at step 1108, a daughter scope or auxiliary scope can be extended from the mother scope. For example, the auxiliary scope 434 can be extended from the endoscope functional module 402. At step 1110, the daughter scope can engage the guide. For example, the auxiliary scope 434 can engage the guide 460 such as to guide extension of the auxiliary scope 434 from the opening 454 of the working channel 448 of the functional module 402.

At step 1112, the daughter scope can engage the arm. For example, the auxiliary scope 434 can engage the arm 462 such as to guide extension of the auxiliary scope 434 from the opening 454 of the working channel 448 of the functional module 402. At step 1114, the scope can be inserted into a channel, passage, or duct. For example, the auxiliary scope 434 can be extended into the sphincter of Oddi, such as to perform an ERCP.

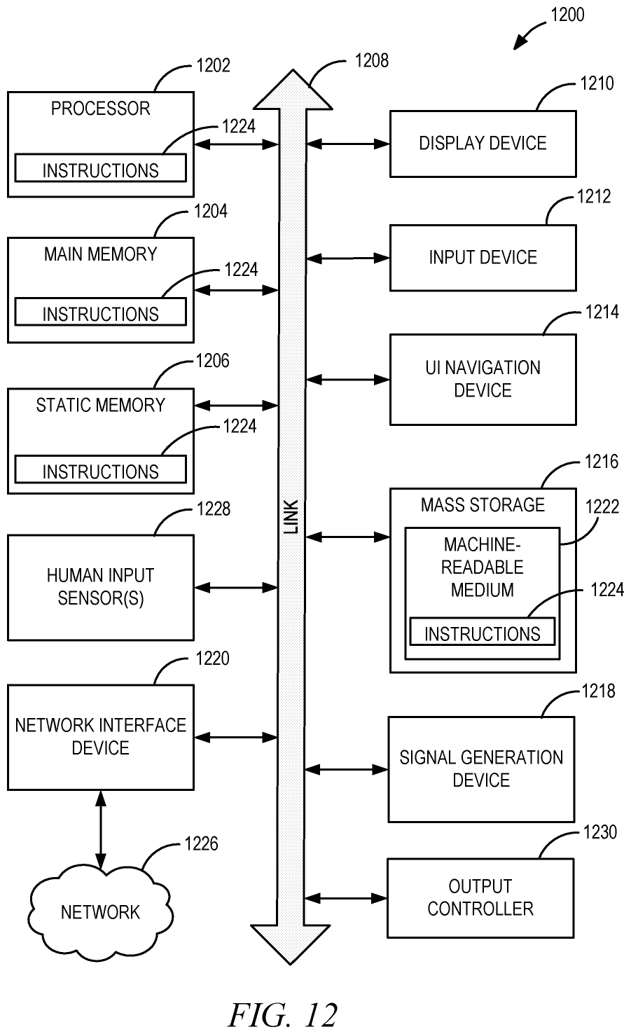

FIG. 12 illustrates a block diagram of an example machine 1200 upon which any one or more of the techniques (e.g., methodologies) discussed herein may perform. Examples, as described herein, may include, or may operate by, logic or a number of components, or mechanisms in the machine 1200. Circuitry (e.g., processing circuitry) is a collection of circuits implemented in tangible entities of the machine 1200 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1200 follow.

In alternative embodiments, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1200 may include a hardware processor 1202 (e.g., a central processing unit (CPU), a graphics processing unit (GPU), a hardware processor core, or any combination thereof), a main memory 1204, a static memory (e.g., memory or storage for firmware, microcode, a basic-input-output (BIOS), unified extensible firmware interface (UEFI), etc.) 1206, and mass storage 1208 (e.g., hard drive, tape drive, flash storage, or other block devices) some or all of which may communicate with each other via an interlink (e.g., bus) 1230. The machine 1200 may further include a display unit 1210, an alphanumeric input device 1212 (e.g., a keyboard), and a user interface (UI) navigation device 1214 (e.g., a mouse). In an example, the display unit 1210, input device 1212 and UI navigation device 1214 may be a touch screen display. The machine 1200 may additionally include a storage device (e.g., drive unit) 1208, a signal generation device 1218 (e.g., a speaker), a network interface device 1220, and one or more sensors 1216, such as a global positioning system (GPS) sensor, compass, accelerometer, or other sensor. The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1202, the main memory 1204, the static memory 1206, or the mass storage 1208 may be, or include, a machine readable medium 1222 on which is stored one or more sets of data structures or instructions 1224 (e.g., software) embodying or utilized by any one or more of the techniques or functions described herein. The instructions 1224 may also reside, completely or at least partially, within any of registers of the processor 1202, the main memory 1204, the static memory 1206, or the mass storage 1208 during execution thereof by the machine 1200. In an example, one or any combination of the hardware processor 1202, the main memory 1204, the static memory 1206, or the mass storage 1208 may constitute the machine readable media 1222. While the machine readable medium 1222 is illustrated as a single medium, the term “machine readable medium” may include a single medium or multiple media (e.g., a centralized or distributed database, and/or associated caches and servers) configured to store the one or more instructions 1224.

The term “machine readable medium” may include any medium that is capable of storing, encoding, or carrying instructions for execution by the machine 1200 and that cause the machine 1200 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices;
magnetic disks, such as internal hard disks and removable disks; magneto-optical disks; and CD-ROM and DVD-ROM disks.

The instructions 1224 may be further transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1220 may include one or more physical jacks (e.g., Ethernet, coaxial, or phone jacks) or one or more antennas to connect to the communications network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1200, and includes digital or analog communications signals or other intangible medium to facilitate communication of such software. A transmission medium is a machine readable medium.

### NOTES AND EXAMPLES

The following, non-limiting examples, detail certain aspects of the present subject matter to solve the challenges and provide the benefits discussed herein, among others.

Example 1 is an endoscope system comprising: a control handle; a primary scope extending from the control handle to a distal portion and configured for insertion into a passage of a patient, the primary scope defining a working channel extending therethrough; an auxiliary scope located at least partially within the working channel and movable to extend from the primary scope; and a guide extendable from the distal portion of the primary scope to guide extension of the auxiliary scope from the primary scope.

In Example 2, the subject matter of Example 1 optionally includes wherein the guide is extendable from the working channel and engageable with the auxiliary scope to guide extension of the auxiliary scope from the primary scope.

In Example 3, the subject matter of Example 2 optionally includes an arm pivotably connected to the distal portion of the primary scope, the arm engageable with the guide to guide extension of the guide from the primary scope.

In Example 4, the subject matter of Example 3 optionally includes wherein the arm is engageable with the auxiliary scope to guide extension of the auxiliary scope from the primary scope.

In Example 5, the subject matter of any one or more of Examples 3-4 optionally include a guide wire extendable from the primary scope, the guide engageable with the guide wire to guide extension of the guide wire from the primary scope.

In Example 6, the subject matter of Example 5 optionally includes wherein the arm is engageable with the guide wire to guide extension of the guide wire from the primary scope.

In Example 7, the subject matter of any one or more of Examples 5-6 optionally include wherein the guide wire is extendable from a second channel located near the working channel.

In Example 8, the subject matter of any one or more of Examples 2-7 optionally include wherein the guide is tapered from a proximal portion to a distal portion.

In Example 9, the subject matter of any one or more of Examples 2-8 optionally include wherein a distal portion of the guide is configured to deliver energy to ablate tissue.

In Example 10, the subject matter of any one or more of Examples 1-9 optionally include a stabilizer connected to the primary scope and configured to extend therefrom to engage an inner intestinal wall.

In Example 11, the subject matter of Example 10 optionally includes wherein the stabilizer includes a plurality of projections extending from the primary scope, each projection configured to engage the inner intestinal wall.

In Example 12, the subject matter of any one or more of Examples 1-11 optionally include an image capture device connected to an outer portion of the primary scope and angled with respect to a longitudinal axis of the primary scope.

In Example 13, the subject matter of any one or more of Examples 1-12 optionally include wherein the primary scope is a duodenoscope and the auxiliary scope is a cholangioscope.

Example 14 is an endoscope system comprising: a primary scope extending from a proximal portion to a distal portion and configured for insertion into a passage of a patient, the primary scope defining a working channel extending therethrough; an auxiliary scope located at least partially within the working channel and movable to extend from the primary scope; and a guide extendable from the distal portion of the primary scope to direct advancement of the auxiliary scope from the primary scope.

In Example 15, the subject matter of Example 14 optionally includes wherein the guide is extendable from the working channel and engageable with the auxiliary scope to guide extension of the auxiliary scope from the primary scope.

In Example 16, the subject matter of Example 15 optionally includes an arm pivotably connected to the distal portion of the primary scope, the arm engageable with the guide to guide extension of the guide from the primary scope.

In Example 17, the subject matter of Example 16 optionally includes wherein the arm is engageable with the auxiliary scope to guide extension of the auxiliary scope from the primary scope.

In Example 18, the subject matter of any one or more of Examples 16-17 optionally include a guide wire extendable from the primary scope, the guide engageable with the guide wire to guide extension of the guide wire from the primary scope.

In Example 19, the subject matter of Example 18 optionally includes wherein the arm is engageable with the guide wire to guide extension of the guide wire from the primary scope.

Example 20 is a method of operating an endoscope system, the method comprising: extending a guide from a distal portion of a primary scope near a working channel of the primary scope; engaging the guide with an arm connected to the distal portion of the primary scope; extending a auxiliary scope from the working channel to engage the guide; guiding extension of the n from the working channel by engaging the guide with the auxiliary scope.

In Example 21, the subject matter of Example 20 optionally includes engaging the arm with the auxiliary scope.

In Example 22, the subject matter of Example 21 optionally includes cannulating a sphincter of Oddi with a tip of the guide.

In Example 23, the subject matter of Example 22 optionally includes extending the auxiliary scope into the sphincter of Oddi following cannulation.

In Example 24, the apparatuses or method of any one or any combination of Examples 1-23 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An endoscope system comprising:
a control handle;
a primary scope extending from the control handle to a distal portion and configured for insertion into a passage of a patient, the primary scope defining a working channel extending therethrough;
an auxiliary scope located at least partially within the working channel and movable to extend from the primary scope; and
a guide extendable from the distal portion of the primary scope to guide extension of the auxiliary scope from the primary scope.

2. The endoscope system of claim 1, wherein the guide is extendable from the working channel and engageable with the auxiliary scope to guide extension of the auxiliary scope from the primary scope.

3. The endoscope system of claim 2, further comprising:
an arm pivotably connected to the distal portion of the primary scope, the arm engageable with the guide to guide extension of the guide from the primary scope.

4. The endoscope system of claim 3, wherein the arm is engageable with the auxiliary scope to guide extension of the auxiliary scope from the primary scope.

5. The endoscope system of claim 3, further comprising:
a guide wire extendable from the primary scope, the guide engageable with the guide wire to guide extension of the guide wire from the primary scope.

6. The endoscope system of claim 5, wherein the arm is engageable with the guide wire to guide extension of the guide wire from the primary scope.

7. The endoscope system of claim 5, wherein the guide wire is extendable from a second channel located near the working channel.

8. The endoscope system of claim 2, wherein the guide is tapered from a proximal portion to a distal portion.

9. The endoscope system of claim 2, wherein a distal portion of the guide is configured to deliver energy to ablate tissue.

10. The endoscope system of claim 1, further comprising:
a stabilizer connected to the primary scope and configured to extend therefrom to engage an inner intestinal wall.

11. The endoscope system of claim 10, wherein the stabilizer includes a plurality of projections extending from the primary scope, each projection configured to engage the inner intestinal wall.

12. The endoscope system of claim 1, further comprising:
an image capture device connected to an outer portion of the primary scope and angled with respect to a longitudinal axis of the primary scope.

13. The endoscope system of claim 1, wherein the primary scope is a duodenoscope and the auxiliary scope is a cholangioscope.

14. A method of operating an endoscope system, the method comprising:
extending a guide from a distal portion of a primary scope near a working channel of the primary scope;
engaging the guide with an arm connected to the distal portion of the primary scope;
extending an auxiliary scope from the working channel to engage the guide;
guiding extension of the n from the working channel by engaging the guide with the auxiliary scope.

15. The endoscope system of claim 14, further comprising:
engaging the arm with the auxiliary scope.

16. The endoscope system of claim 15, further comprising:
cannulating a sphincter of Oddi with a tip of the guide.

17. The endoscope system of claim 16, further comprising:
extending the auxiliary scope into the sphincter of Oddi following cannulation.

18. An endoscope system comprising:
a primary scope extending from a proximal portion to a distal portion and configured for insertion into a passage of a patient, the primary scope defining a working channel extending therethrough;
an auxiliary scope located at least partially within the working channel and movable to extend from the primary scope; and
a guide extendable from the distal portion of the primary scope to direct advancement of the auxiliary scope from the primary scope.

19. The endoscope system of claim 18, wherein the guide is extendable from the working channel and engageable with the auxiliary scope to guide extension of the auxiliary scope from the primary scope.

20. The endoscope system of claim 19, further comprising:
an arm pivotably connected to the distal portion of the primary scope, the arm engageable with the guide to guide extension of the guide from the primary scope.

21. The endoscope system of claim 20, wherein the arm is engageable with the auxiliary scope to guide extension of the auxiliary scope from the primary scope.

22. The endoscope system of claim 20, further comprising:
a guide wire extendable from the primary scope, the guide engageable with the guide wire to guide extension of the guide wire from the primary scope.

23. The endoscope system of claim 22, wherein the arm is engageable with the guide wire to guide extension of the guide wire from the primary scope.
